(19) United States
(12) Patent Application Publication
(10) Pub. No.: US 2025/0152046 A1
(43) Pub. Date: May 15, 2025

(54) WEARABLE DEVICE MEASURING BLOOD GLUCOSE LEVELS BASED ON GLUCOSE REFLECTIVITY

(71) Applicant: Bruce Landy, Parker, CO (US)

(72) Inventor: Bruce Landy, Parker, CO (US)

(21) Appl. No.: 18/614,142

(22) Filed: Mar. 22, 2024

Related U.S. Application Data
(60) Provisional application No. 63/599,302, filed on Nov. 15, 2023.

(51) Int. Cl. A61B 5/145; A61B 5/00; A61B 5/1455

(52) U.S. Cl. CPC A61B 5/14532; A61B 5/1455; A61B 5/6803; A61B 5/681; A61B 5/743

(57) ABSTRACT

The blood glucose detector (BGD) is placed on user's wrist, ear, nose, or other skin surface, at a NIR light infiltration location to detect arterial blood flow. Earlier-obtained baseline BG data is obtained via BGD and conventionally-obtained BG data. The user-wearable housing or body-mounted BGD system includes processor, memory, and NIR transmitter/sensor. BG baseline data is stored in memory. The NIR transmitter/sensor disposed on infiltration location. In detection, transmitter generates light in predetermined narrow band within NIR range 1350-1800 nm. Sensor detects reflected light from arterial BG as then-detected BG signal. Memory stores a subcutaneous interference factor (SIF) contributory value for NIR narrow band. Onboard processor (or smart phone APP) correlates the detected BG signal with the baseline as modified by the SIF value and generates displayable BG level to user via user-wearable housing or the user interface display on the smart phone as connected to body-mounted BGD system.

This application claims priority from provisional patent application Ser. No. 63/599,302, filed Nov. 15, 2023, the contents of which is incorporated herein by reference thereto. The contents of U.S. Pat. No. 11,701,035, for a Noninvasive Blood Glucose Detector and Method Using IR, is also incorporated herein by reference thereto. The present invention relates to a wearable device that measures the reflectivity of glucose in order to obtain blood glucose levels and display the same to the user wearing the blood glucose device (herein “BGD”).

### BACKGROUND

The '035 patent relates to a blood glucose (herein “BG”) detection system and a method using infrared (IR) light in a “1550 nm bandwidth range” comprising 1085 nm to 2015 nm in order to provide a measurement of the user's blood glucose level. The '035 patent further explains that in one embodiment, the IR system may have an IR transmitter and an IR sensor on a single structural member wherein the transmitter/sensor is angled and configured to transmit IR light and sense IR light reflected off the “blood flow in the finger web or [ear] antihelix.”

The '035 patent also explains that the user inputs his or her conventionally obtained baseline blood glucose or BG data into the BGD (alternatively, a healthcare provider inputs the conventionally obtained BG data into the BGD). The to-be-input BG data is earlier obtained from a conventional device prior to the initialization sub-program in the blood glucose device (BGD). Once the conventionally acquired and to-be-input BG data is obtained, the BG detector on the user's finger web or ear antihelix obtains a corresponding “detected BG signal” based upon the 1550 nm bandwidth NIR signal transmitted and reflected by blood in the body part. The processor uses (a) the conventionally acquired/inputted BG data and (b) the then-detected BG signal to generate a “baseline detected BG signal.” The baseline BG signal level is stored in the BGD memory at the initialization phase. During the initialization, the then-detected BG signal is correlated or matched to the conventionally acquired input BG data, stated otherwise, the system is normalized by comparing the then-detected BG signal with the conventionally acquired BG data.

After initialization, when the user wants to determine his or her blood glucose level, the user places the BGD on the user's finger web or ear antihelix (preferably at the predetermined web/antihelix position), the BGD transmits NIR light in the NIR 1550 nm range (per the '035 patent, the 1085-2015 nm range), the BGD sensor senses the blood reflected or transmitted light, and compares the then-detected BG signal to the baseline BG signal. The comparison generates a normalized BG signal because the then-acquired BG signal is compared to the BG baseline and adjusted to represent the then-current blood glucose level. The user is presented, via a display, the normalized then-current blood glucose level or a displayable representative BG value or BG indicator.

In connection with the present wearable device invention, the “NIR blood glucose illumination range” (sometimes referred to as the “NIR 1550 range” or “NIR sensory range”) is 1350-1800 nm, which NIR range is narrower than the '035 patent NIR sensory range of 1085-2015 nm.

### SUMMARY OF THE INVENTION

In the present invention, the measured and detected BG signal is the reflected and/or transflected light from the glucose molecules in the user's blood stream and the amount reflected/transflected optical signal is compared to a blood glucose earlier taken by direct blood sampling. The transmitted and reflected NIR sensory range is 1350-1800 nm. The earlier-acquired conventionally obtained BG sample data is used for registering a baseline BG data or level in the BGD which is stored in the BGD memory store. After sufficient sampling of both the reflected/transflected optical signal and earlier-acquired direct blood sample data, the correlation between the data points can be produced by the BGD system. The measured reflected/transflected optical signal can be used can compared against the earlier-acquired “baseline detected BG signal” in the wearable device having similar data processing components as explained in the '035 patent. The processor and memory uses a BG baseline data and compares that baseline data to the then obtained BG reflected/transflected optical detected signal. The resultant normalized BG signal is a representation of the then-currently acquired BG level and the representative BG level is displayed to the user as a BG indication of the then-current condition of the user's glucose.

One embodiment of the BGD is a detector adapted to be placed on a user's wrist or other skin surface at a near infrared (NIR) light infiltration location such that arterial blood flow is detected at the wrist or other skin surface by the NIR light. The BGD is used in conjunction with an earlier obtained baseline blood glucose (BG) data set representing an earlier conventionally obtained blood glucose data. The BGD includes a user-wearable housing encapsulating a processor, a processor-coupled memory, a NIR transmitter and a NIR sensor. The memory stores the baseline BG data set. In one embodiment, the user-wearable housing and the NIR transmitter are disposed on the infiltration location of the user's wrist or other skin surface in a detection mode. In this detection mode, the NIR transmitter generates NIR light in a predetermined narrow transmission band. This NIR narrow band is within a NIR sensory bandwidth of 1350-1800 nm. A non-limiting example of a narrow NIR band is 1400 nm. The NIR sensor detects reflected light from arterial blood glucose representing a then-detected BG signal. The memory also stores a predetermined subcutaneous interference factor (SIF) contributory value for the NIR light narrow transmission band. The processor correlates the detected BG signal with the baseline BG data set as modified by the SIF value and generates a displayable blood glucose level for the user. In this embodiment, the user-wearable housing has a user interface (UI) display. The UI display is coupled to the processor and displays, during the detection mode, the displayable blood glucose level to the user enabling a user comparison with a healthcare recommended blood glucose level.

A further aspect of the present invention includes details of the predetermined SIF value. This SIF value is stored in the memory for the particular narrow transmission band and accounts for a predetermined glucose reflectivity, a predetermined absorption spectrum of water, and a predetermined reflectivity of skin. The predetermined SIF value is an algorithmic amalgamation of the predetermined glucose reflectivity, the predetermined absorption spectrum of water, and the predetermined reflectivity of skin. One algorithm for the predetermined SIF value includes a reduction accounting for the predetermined reflectivity of skin.

In connection with the NIR narrow band, the NIR sensory bandwidth of 1350-1800 nm encompasses (a) at a NIR light transmission centerpoint 1350 nm, a spectral line half width wavelength Δλ no less than 1300 nm, and (b) at a NIR light transmission at centerpoint 1800 nm, a spectral line half width wavelength Δλ no more than 1850 nm.

A further embodiment handles infiltration data. The BGD may include, in memory, infiltration data obtained at a time of obtaining the baseline BG level accounting for, at the NIR light narrow transmission band, the absorption spectrum of water and the reflectivity of skin. The processor, at that initialization time, stores infiltration data in memory. Further, at the time of acquiring the then-detected BG signal obtaining, the BGD also obtains then-detected infiltration data. The BGD processor compares the earlier stored infiltration data to the then-current infiltration data, and issues an alarm in the user's UI if the differential between the stored and the then-detected infiltration data exceeds a predetermined positional value.

The invention also includes a method for detecting BG on a user's wrist or other skin surface at the NIR light infiltration location. During an initialization phase, the BGD obtains a baseline BG data set correlated to a then-conventionally obtained BG data. As noted, the BGD includes a user-wearable housing, a housed processor and processor-coupled memory, a NIR transmitter, and a NIR sensor. The NIR transmitter generates light in a predetermined narrow transmission band which narrow band is within a larger NIR sensory bandwidth of 1350-1800 nm. The method stores in memory the baseline BG data set. In a post-initialization detection mode, the user-wearable housing and the NIR transmitter is placed on the infiltration location of the user's wrist or other skin surface. Further and later on, in the detection mode, the BGD transmits NIR light (at the narrow band) into the infiltration location. The BGD detects, with the NIR sensor, reflected NIR light from arterial blood glucose and the resulting received signal represents a then-detected blood glucose (BG) signal. Prior to initialization and detection, a predetermined subcutaneous interference factor (SIF) contributory value for the NIR light narrow transmission band is stored in memory. The BGD processor correlates the detected BG signal with the baseline BG data set as modified by the SIF contributory value. As a result, the BGD generates a displayable blood glucose level for the user based upon the correlated and signals as modified by the SIF contributory value.

Further enhancements of the BG detection method include a predetermined SIF value which accounts for a predetermined glucose reflectivity, a predetermined absorption spectrum of water, and a predetermined reflectivity of skin. Further, the predetermined SIF value is an amalgamation of the predetermined glucose reflectivity, the predetermined absorption spectrum of water, and the predetermined reflectivity of skin. More precisely, the predetermined SIF value includes a reduction accounting for the predetermined reflectivity of skin. As discussed earlier, the NIR sensory bandwidth of 1350-1800 nm encompasses (a) at a NIR light transmission centerpoint 1350 nm, a spectral line half width wavelength Δλ no less than 1300 μm, and (b) at a NIR light transmission at centerpoint 1800 nm, a spectral line half width wavelength Δλ no more than 1850 nm.

Another embodiment stores, in the memory, an infiltration data at a time of obtaining the baseline BG level accounting for, at the NIR light narrow transmission band, the absorption spectrum of water and the reflectivity of skin. At the time of acquiring the then-detected BG signal, the system obtains a then-detected infiltration data and compares the stored infiltration data to the then-detected infiltration data. If the data exceeds a predetermined positional value, the BGD issues an alarm based on the differential between stored and detected infiltration data.

A further embodiment of the present invention is a two-piece system consisting, in general, of a BG detector and a wirelessly coupled smart phone. The BG detection system includes a body-mountable BG detector and a communicatively linked smart phone. The body-mountable detector is placed on a user's ear skin surface or nose skin surface at a near infrared (NIR) light infiltration location such that arterial blood flow is detected at the user's ear skin surface or nose skin surface by the NIR light. The smart phone has a memory storing therein an earlier obtained baseline BG data set representing an earlier conventionally obtained BG data.

In this two-piece BG system, the body-mountable detector includes a number of items. An onboard unit is mounted on or in one body-mountable structure from a group of body-mountable structures including: (a) an eyeglass temple coupled to one eyeglass, (b) an eyeglass nose bridge coupled to another eyeglass, (c) a clip-on earring body, (d) a stud mounted earring body, and (e) a stud mounted nose body. Each respective body-mountable structure has a corresponding skin-side surface from the group of skin-side surfaces consisting of: (i) for the eyeglass temple, a temple-ear skin-side surface; (ii) for the eyeglass nose bridge, a nose bridge skin-side surface; (iii) for the clip-on earring, an clip earring ear skin-side surface; (iv) for the stud mounted earring, a stud earring ear skin-side surface; and (v) for the stud mounted nose body, a nose skin-side surface.

The onboard unit has a NIR transmitter and a NIR sensor encased in the onboard unit. The NIR transmitter and NIR sensor is located on the corresponding skin-side surface of the respective body-mountable structure. Also, the onboard unit has a control processor, coupled to an onboard power supply, to initiate NIR light transmission via the NIR transmitter and enable sensing of the return NIR light via the NIR sensor. The control processor also enables wireless communications via a wireless communications port with the smart phone. The NIR transmitter generates NIR light in a predetermined narrow transmission band which narrow band is within a NIR sensory bandwidth of 1350-1800 nm.

To facilitate BG detection, in a detection mode, the NIR transmitter and sensor, both of which are located on the corresponding skin-side surface of the onboard unit's body-mountable structure, are placed or disposed on the infiltration location of the user's ear skin surface or nose surface. The NIR sensor then detects reflected light from arterial blood glucose representing a then-detected blood glucose signal. The control processor sends the then-detected blood glucose signal to the communicatively coupled smart phone.

On the smart phone side, the phone has a processor coupled to a memory. The phone also has at least a user interface (UI) display and a communications module. The communications module enables wireless communications with the onboard unit. The phone memory stores a predetermined subcutaneous interference factor (SIF) contributory value for the NIR light narrow transmission band. The processor in the phone correlates the then-detected BG signal with the baseline BG data set as modified by the SIF value and generates a displayable BG level for the user. The processor, during the detection mode, displays a representative BG level to the user enabling a user comparison with a healthcare recommended blood glucose level.

The SIF value, stored in the memory for the narrow transmission band, accounts for a predetermined glucose reflectivity, a predetermined absorption spectrum of water, and a predetermined reflectivity of skin. Further, the SIF value is an amalgamation of the predetermined glucose reflectivity, the predetermined absorption spectrum of water, and the predetermined reflectivity of skin. This algorithmic process for SIF value includes a reduction accounting for the predetermined reflectivity of skin. The narrow band is within the NIR sensory bandwidth of 1350-1800 nm encompasses and the spectral line half width wavelengths Δλ above and below the 1350-1800 nm sensory bandwidth are accounted for because the narrow band is a centerpoint peak high intensity of the NIR transmitter.

In another enhancement, the BG detection system smart phone includes a UI alarm. The detection system stores in the memory an infiltration data at a time of obtaining the baseline BG level accounting for, at the NIR light narrow transmission band, the absorption spectrum of water and the reflectivity of skin. At the time of acquiring the then-detected BG signal, the processor compares the stored infiltration data with the infiltration data, and issues an alarm via the UI alarm if the differential therebetween stored and detected infiltration data exceeds a predetermined positional value.

One of the several objectives of the present invention is to provide a blood glucose device (BGD) operable on a user's wrist or other skin surface.

It is another objective of the present invention to provide a BGD as a body-mountable detector. This body-mountable detector may use eyeglasses, a clip-on earring, an earring stud or a stud mounted nose body. These body-mountable detectors carry an onboard unit. The onboard unit has skin-side surfaces from which NIR transmitters and NIR sensors are used to detect BG signals. For example, skin-side surfaces include: for an eyeglass temple, a temple-ear skin-side surface; for an eyeglass nose bridge, a nose bridge skin-side surface; for a clip-on earring, a clip earring ear skin-side surface; for the stud mounted earring, a stud earring ear skin-side surface; and for the stud mounted nose body, a nose skin-side surface.

It is a further objective to account for a subcutaneous interference factor (SIF) contributory value to be applied to the sensed then-detected BG return signal for the NIR light narrow transmission band transmitted by the NIR transmitter. Dependent upon the selected narrow band wavelength generated by the NIR transmitter, the SIF value changes. As an example, if the NIR transmitter generates a narrow band NIR light at 1450 nm, the SIF adjustment is less than if the NIR transmitter generates a NIR light at 1750 nm because the SIF adjustment accounts for a higher level of skin reflectivity at the 1750 nm wavelength as compared to the skin reflectivity at 1450 nm.

An additional objective of the present invention accounts for a SIF value wherein the SIF is a percentage of the returned/sensed NIR signal, taking into account the % of Glucose Reflectivity at the designated narrow NIR transmitted/returned band plus the % of Absorption Spectrum of Water at that narrow band and as adjusted by subtracting % Reflectivity of Skin at that narrow band. Studies have shown that the reflectivity of skin in the NIR sensory range of 1350-1800 nm varies as follows (as an example): 1350 nm is 20%; 1450 nm is 5%; 1550 nm is 10%; 1650 nm is 12% and at 1800 nm is 10%. Also, the reflectivity of glucose varies as follows (as an example): 1350 nm is 30%; 1450 nm is 42%; 1550 nm is 70%; 1650 nm is 68% and at 1800 nm is 55%. The objective in the present invention is to use the narrow transmission band with the larger sensory band of 1350-1800 nm. The variability of water absorption also complicates use of the subcutaneous interference factor (SIF),

A further objective of the present invention is to use earlier acquired infiltration data (acquired when the BG baseline is acquired during initialization) and to compare that infiltration data to the proposed location for detecting BG returned NIR light, that is at the then-detected BG signal data. This objective is accomplished by accounting for the % absorption of water and removing the % glucose reflectivity value. For example, when the NIR transmitter has a centerpoint or peak intensity at 1350 nm, the absorption spectrum of water is 4%, and the reflectivity of skin 20%, therefore the infiltration factor (IF) value is 24% of the returned, sensed NIR light signal because the water absorbed 4% of the NIR light and reflectivity of skin reflected 20% of the NIR light signal. This IF contributory adjustment ignores the glucose data present in the returned, sensed NIR signal.

### BRIEF DESCRIPTION OF THE DRAWINGS

Further objects and advantages of the present invention can be found in the following description of embodiments of the invention when taken in conjunction with the following drawings.

FIGS. 1A, 1B, 2A and 2B are illustrative prior art graphs for, a wide NIR bandwidth range for the spectral reflectance of skin, a narrower NIR bandwidth range for the spectral reflectance of skin, a wide NIR bandwidth range for the absorption of NIR light in water, and a wide NIR bandwidth range for the spectral reflectivity of glucose, respectively.

FIG. 3 diagrammatically illustrates a watch-based blood glucose detector (BGD). It should be noted that the watch-based BGD could be a simple bracelet. Hence these terms are interchangeable.

FIG. 4 diagrammatically illustrates a patch-configured BGD.

FIG. 5A diagrammatically illustrates a medallion-configured BGD.

FIGS. 5B and 5C diagrammatically illustrate two eyeglass-configured BGDs. FIG. 5B illustrating a nose bridge mounted or embodied BGD and FIG. 5C illustrating a temple mounted or embodied BGD. The BGD is an onboard unit which interacts with: for the eyeglass temple (FIG. 5B), a temple-ear skin-side surface and for the eyeglass nose bridge (FIG. 5C), a nose bridge skin-side surface. Also, FIG. 5B diagrammatically illustrates a smart phone which is used in conjunction with the eyeglasses of FIG. 5C, and earring of FIG. 5D and the earring or nose element of FIG. 5E.

FIG. 5D diagrammatically illustrates a clip-on earring BGD. The BGD is an onboard unit which interacts for the clip-on earring (FIG. 5D), a clip earring ear skin-side surface.

FIG. 5E diagrammatically illustrates either a stud earring BGD or a studded nose body BGD. The BGD is an onboard unit which interacts for the stud mounted earring (FIG. 5E), a stud earring ear skin-side surface; and for the stud mounted nose body (also FIG. 5E), a nose skin-side surface.

FIG. 6 diagrammatically illustrates, in block diagram form, the blood glucose detector and electrical components associated therewith. It should be noted that many, if not all, of the electronic components discussed herein may be configured on one or more ICs. As discussed later, in the smart phone embodiments, portions of the BGD system in FIG. 6 are disbursed in the onboard unit of the body-mountable structure and other parts are enabled by the smart phone processor and memory.

The wearable BGD may be a watch-configured BGD, a medallion-configured BGD, a patch-like BGD, or in a mobile phone APP driven system, the BGD is on a user's eyeglasses frame (proximate an ear or the nose) or in a nose ring platform or an earring platform.

### DETAILED DESCRIPTION OF EMBODIMENTS OF THE INVENTION

In the present invention, the measured and detected BG signal is the reflected and/or transflected light from the glucose molecules in the user's blood stream and the amount reflected/transflected optical signal is compared to a blood glucose data earlier taken by direct or conventionally obtained blood sampling. This conventional earlier-acquired BG sample data is input into the BGD system at or near the same time the BGD senses the reflected NIR sensory range, generally encompassing 1550 nm light range, from the blood glucose beneath the wearable BGD. The transmitted and reflected “NIR sensory range” is 1350-1800 nm. The wearable BGD may be a watch-configured BGD, a medallion-configured BGD, a patch-like BGD, or in a mobile phone APP driven system, the BGD is on a user's eyeglasses frame (proximate an ear or the nose) or in a nose ring platform or an earring platform.

At the initialization phase, the conventionally acquired BG sample data is input into the BGD system at or near the same time then-acquired BG data is sensed by the BGD system within the NIR sensory range (1350-1800 nm). During this initialization phase, the BGD on-board processor normalizes or correlates the then-acquired BG data to the conventionally acquired BG sample data and creates a “baseline” BG data point or signal. Future-acquired BG NIR light signal data, within the NIR sensory range, is compared to the stored baseline BG data point or signal level.

In some embodiments, the on-board BGD processor normalizes the then-acquired BG signal to the stored BG baseline. In other smart phone APP-based embodiments, the APP normalizes the signals and stores BG baseline in the phone.

To account for subcutaneous interference with the reflected NIR sensory range light, either (a) the then-acquired BG signal is adjusted by a first subcutaneous interference factor or (b) the normalized BG signal is adjusted by a second subcutaneous interference factor. In general, the subcutaneous interference factor (SIF) accounts for reflectivity of water, absorption of water in the NIR sensory range, the spectral reflectivity of glucose and the spectral reflectance of skin. Dependent upon the selected wavelength generated by the NIR transmitter, the SIF value changes. In other words, if the NIR transmitter generates a NIR light at 1450 nm, the SIF adjustment is less than if the NIR transmitter generates a NIR light at 1750 nm because the SIF adjustment must account for a higher level of skin reflectivity at the 1750 nm wavelength. The first SIF value is correlated to the raw, then-acquired BG signal whereas the second SIF value is correlated to the normalized then-acquired BG signal (normalized to the stored BG baseline level).

The then-acquired, normalized signal and SIF adjusted BG signal provides an indication or a representation of the user's BG level at the then-current time the BGD senses blood glucose in the NIR sensory range. The BGD display may show the user a simple numerical value (1-10, wherein 10 is the best BG indicator) or a more complex numerical value (level/ranges like 100-125, 125-150, 150-200, and 200-300 mg/dl (these range brackets may be conventional BG range brackets) or something simpler such as GOOD, OK, or BAD or green, blue or red (red indicating an unacceptable high BG level).

The near infrared (NIR) light transmitter is mounted on the backside or skin-side of the watch-like structure such that NIR light is directed to and through the user's adjacent skin. The NIR light sensor is also mounted on the backside or skin-side of the watch-like structure. Possibly, the transmitter also operates as a sensor. The NIR sensory wavelength band or range is described above. Other embodiments of the BGD are discussed later.

Operationally, the emitted near infrared (NIR) light sent from the transmitter though the user's skin is partially reflected by glucose, and the glucose-reflected light is partially prevented from further through-arterial-transmission by glucose. The glucose-reflected light is sensed by the backside sensor in the watch-like structure or other BGD system. The higher the concentration of glucose in the blood, the higher the reflected transmission of the near infrared light. In other words, some reflected NIR light is further blocked by glucose as that reflected light passes through the biologic fluids and membranes to the receptive sensor. The term glucose-reflected light includes refracted and reflected NIR light as effected by the glucose molecules. The graphs discussed below establish that the greater the amount of reflection of the near infrared light indicates blood glucose levels. The receptive NIR sensor detects the through-transmission and glucose-reflected NIR light in the sensory bandwidth. The graphs show that the interaction of skin and bodily fluids can be accounted for when measuring glucose levels via glucose-reflected light.

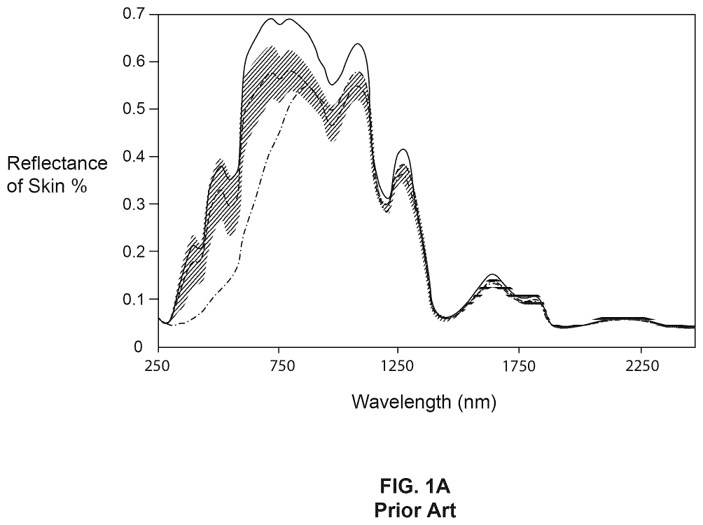

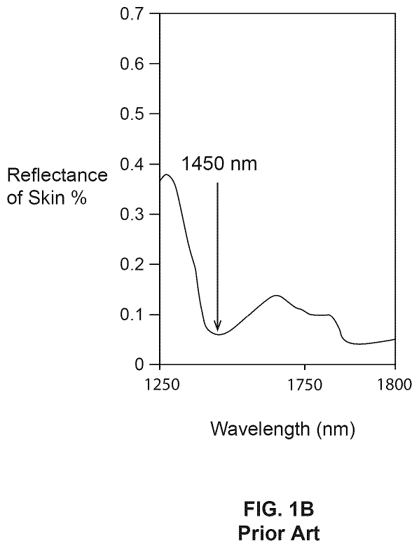

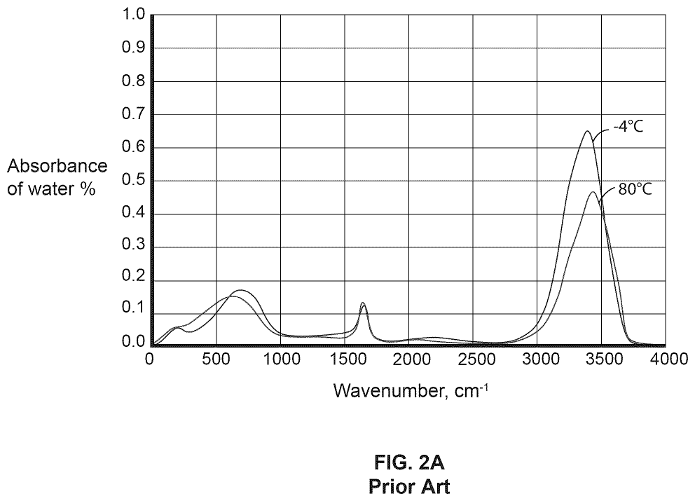

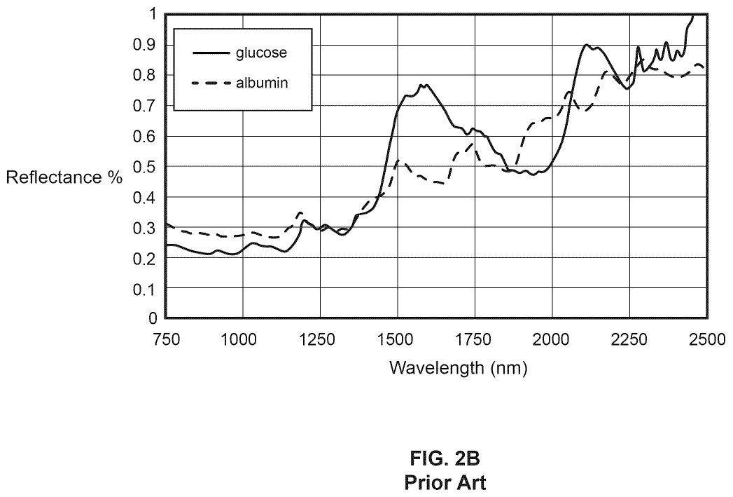

FIGS. 1A, 1B, 2A and 2B Graphs. The prior art graphs in FIGS. 1A, 1B, 2A, and 2B generally show reflected/transflected optical signals over a wide range of the NIR light spectrum. However, the BG detector must account for (1) the reflectivity of water, since about 70% of arterial blood is water; (ii) absorption of the light in the NIR sensory range by water; (iii) the spectral reflectivity of glucose; and (iv) the spectral reflectance of skin. For these reasons, the subcutaneous interference factor (SIF) or value in the present invention accounts for the different subcutaneous interactions with the NIR transmitted, reflected and sensed light from the arterial blood flow. The reflected and sensed NIR light is effected by the percentage of absorption and reflection of the selected NIR light wavelength and the sensed signal at the NIR receiver senses higher levels of reflected light and lower levels of absorbed light as the light passes through the subcutaneous structures.

Reflectivity of Water. The reflectivity of water in the NIR sensory range of 1350-1800 nm is nil. According to “SEOS Classification Algorithms and Methods,” the reflectivity of water in a NIR range 1375-1800 nm (1.3-1.8 μm) is near zero. The maximum reflectivity of clear water is about 15% at about 500-600 nm (0.5-0.6 μm) (outside the NIR sensory range). Hence, since blood is about 70% water, that is, mostly water, there should be virtually no NIR signal in the sensory range of 1350-1800 nm reflected back signal from the water in the blood stream. The SEOS Classification Algorithms article states “Generally, water only reflects in the visible light range. As water has almost no reflection in the near infrared range it is very distinct from other surfaces.”

The article entitled “A Real Time Vein Detection System” by K. Ahmed, states that “imaging utilizing Near Infrared (NIR) has turned out to be an exceptional instrument in restorative diagnostics. For example, NIR is used in Optical Intelligibility Tomography equipped for 3D imaging with high determination keeping pace with low-control microscopy, in measuring bloodstream, blood volume, O2 utilization, re-oxygenation rates and muscle recuperation time in muscle, in the noninvasive evaluation of cerebrum work and other medical applications.” This article indicates that NIR illumination of in vivo skin around 740-940 nm (outside the NIR sensory range) has the capability to infiltrate 5 mm into the skin tissue. However, the authors in this study were focused on colorizing the reflected images, mainly for vein location and detection. The article states “The color of objects is determined by the percentage of absorption and reflection of different wavelengths projected toward that object, we can see red a ball red because it absorbs the wavelengths of blue and green while reflecting red.”

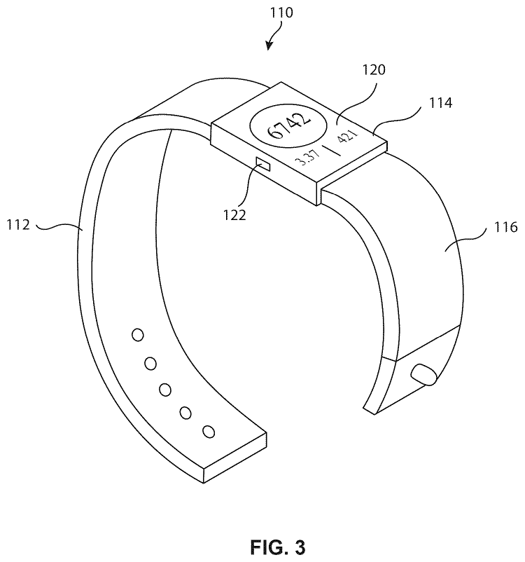

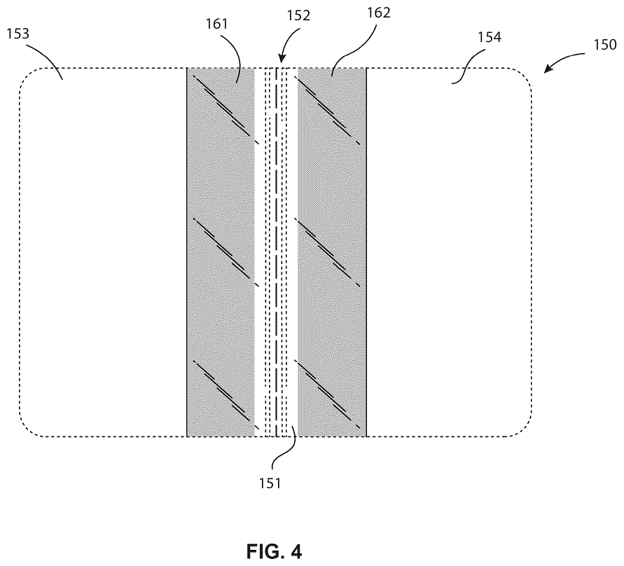

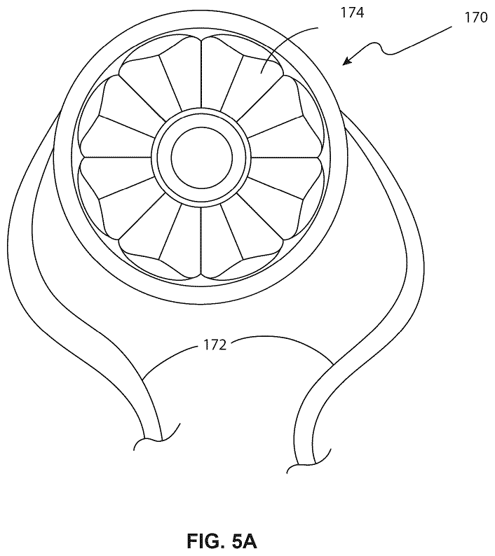

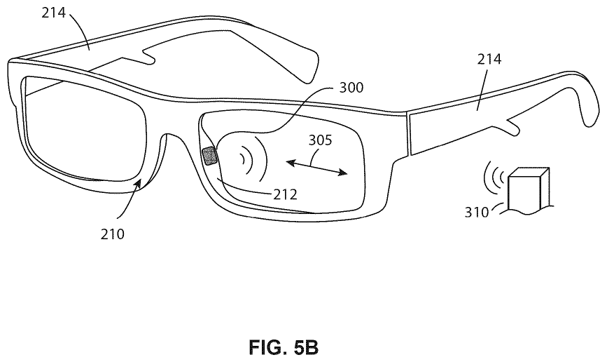

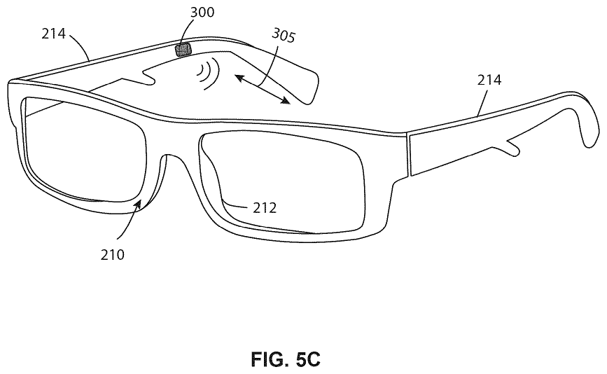

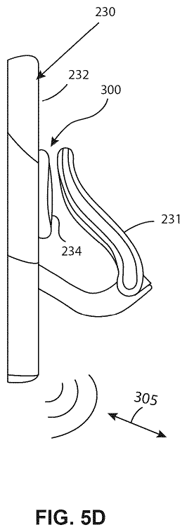

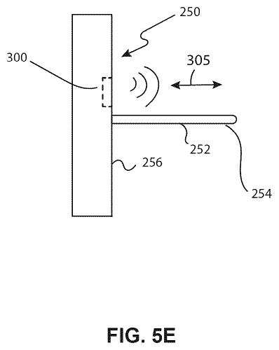

The Subcutaneous Infiltration. Ahmed indicates that 740-940 nm light penetrates 5 mm into the skin. Therefore, the present BGD invention operating at a certain NIR wavelength within the sensory NIR range would subcutaneously infiltrate the user's skin in a similar manner. Hence, BG signals acquired by a watch-based or watch-configured BGD (FIG. 3) may more accurately detect and inform the user of his or her BG level than a medallion-configured BGD (FIG. 5A). The accuracy of a patch-configured BGD (FIG. 4) depends on where the patch is attached to the user's body, that is, the body location. The accuracy of the earring-based BGD should be accurate since the BGD sensor on the skin-side of the earring (FIGS. 5D (clip-on) and 5E (stud)) is at a fixed location. The same accuracy of the nose-ring application (FIG. 5E (stud)) should also be relatively accurate. The eyeglass mounted BGD sensors (FIGS. 5B and 5C) are subject to repositioning by the user but positional accuracy can be achieved as explained later.

The present wearable device BGD is different than the system in the Ahmed article. The present invention is distinguished from the Ahmed article because the Ahmed study illuminated vein-bound “hemoglobin [in] the blood tissues engrossed [by] Near Infrared (NIR) illuminated light and [sensed by a] night vision camera [ ] to capture the scene and enhance the vein pattern clearly using Contrast Limited Adaptive Histogram Equalization (CLAHE) method.” The Ahmed study teaches away from the present invention because Ahmed reports “standard lens camera cannot view the NIR images because it requires an IR none-blocking filter to allow it to view this range from the electromagnetic spectrum.” Ahmed enhances the captured reflected image data and uses a Bi-Histogram Equalization (BBHE) procedure wherein the images are extracted to create multiple sub-images and two consecutive parts of these images are averaged to preserve the average brightness of the input image as an output image. The Ahmed article illustrates a comparison of the output results among BBHE, RMSHE & Recursive Sub Image Histogram Equalization (RSIHE). Hence, the Ahmed methodology and system is very different than the present invention.

Absorption Spectrum of Water (FIG. 2A). Regarding the absorption spectrum of water, the article entitled “Water Structure and Science Water Absorption Spectrum” discloses a peak absorption of water, about 15% absorption, at a NIR 1650 nm wavelength. The article has a graph showing a peak of water's attenuated total reflectance (ATR) measured with infrared spectroscopy at 1655-1750 nm, about 15% absorption at that NIR level. FIG. 2A replicates relevant portions of the graph in the light range 1000-2500 nm where the absorption measurements for liquid water at −4 C degrees and liquid water at 80 C degrees are substantially the same. The NIR sensory range used in connection with the present invention is much narrower than the larger, graphically presented, range of 1000-2500 nm in the Water Structure article.

The Water Structure article states “The near-infrared (NIR) bands (at about λ 970-1940 nm) are suited to rapid non-destructive water determination.” The article further provides data for water absorption in the 1,000-2,000 nm range (replicated in FIG. 2A) which varies from a low of about 3% absorption to an absorption spike of about 13-15% at about 1650 nm. The Water Structure article used Attenuated Total Reflectance (ATR) infrared spectroscopy to measure water absorption. In general, the ATR sampling methodology enables the direct examination of solid or liquid samples without further preparation. The sampling method utilizes total internal reflection to generate an evanescent wave that penetrates the sample, providing valuable molecular information. ATR is used with Fourier transform infrared (FTIR) spectroscopy (ATR-FTIR spectroscopy) as it enables solids and liquid samples to be analyzed neatly-simplifying the measurement of virtually all substances.

The present wearable device BGD is different than the system in the Water Structure article. The Water Structure article processed NIR sensed data using complex ATR spectroscopy. This NIR data process is very different than the differential and SIF adjusted processing used in connection with the present invention. Not only is the computer processing in the present invention much simpler (computing a difference or differential value and adjusting for SIF values at the generated NIR light), but also the sensory equipment in the Water Structure article used spectral scanned ATR data and a Fourier transform process the acquired infrared spectral scan.

Spectral Reflectivity of Glucose (FIG. 2B). Reflectivity of glucose is also be taken into account in the inventive BGD system and method. In contrast to absorption of NIR light by water, the spectral reflectivity of glucose is discussed in the article titled “Determination of Concentrations of Glucose and Human Serum Albumin in Mixtures in Phosphate-Buffered Solution by Near-Infrared Spectroscopy,” by M. Ogawa and T. Yamakoshi. The reflectivity of glucose at 1500-1625 nm reflects about 70-75% of the NIR light. The Ogawa/Yamakoshi study used powdered glucose and human serum albumin samples in a phosphate-buffered solution to measure glucose reflectivity.

Ogawa/Yamakoshi's data, replicated in FIG. 2B, shows, at 1000 nm, glucose reflectance is about 23% and at 2000 nm, glucose reflectance is about 50%. The dark line in FIG. 2B shows glucose reflectance whereas the dashed line in FIG. 2B shows albumin reflectance. The Ogawa/Yamakoshi article shows, in the NIR sensory range of the present invention (1350-1800 nm), at 1350 nm, the glucose reflectance is about 35%; at 1500 nm, the glucose reflectance is about 60%; at 1750 nm, the glucose reflectance is about 60%; and at 1800 nm, the glucose reflectance is about 50%.

The article explains “Near-infrared (NIR) absorbance spectra of glucose and human serum albumin (HSA) mixtures in phosphate-buffered solution (PBS) (pH=7.4) were investigated as the basis of reagent less blood constituent measurements . . . . Differential absorbance spectra taken relative to the spectrum of a reference PBS were measured in the wavelength range of 750-2,500 nm.” The “differential absorbance spectra” discussed in the Ogawa/Yamakoshi article indicates that different NIR wavelengths (1750-2500 nm) were sensed and monitored over a wide spectrum of reflected/returning light signals. Ogawa/Yamakoshi refers to “differential spectra” to detect BG. Ogawa/Yamakoshi used a Fourier transform near-infrared (FT-NIR) spectrometer for absorbance measurements. The article further explains “we have recently proposed a novel noninvasive method of in vivo measurement of blood glucose concentration using an NIR multiwavelength photo plethysmogram (PPG), which was named pulse glucometry. The measurement principle of pulse glucometry is based on the fact that the glucose molecule has absorption bands in the NIR region and the PPG is derived from the arterial volume change of the blood vessel at the measurement site (e.g., fingertip).” Hence, the Ogawa/Yamakoshi transmitted pulses, formed by a series of NIR transmissions, having different wavelengths, into the patient's body and then the system, processing reflected light, engaged a spectral analysis on the returned or reflected NIR signals. Co-author Yamakoshi better explains the system in the Ogawa/Yamakoshi Article (“Determination of Concentrations of Glucose”) in the Yamakoshi '983 patent disclosure.

U.S. Patent Publication no. 20060063983 to Yamakoshi discloses a blood glucose (BG) sensor wherein the transmitter generates NIR (near infrared) light having two different wavelengths which are sequentially emitted into the skin over a 20-cycle period. There are two embodiments in the Yamakoshi '983 patent disclosure. In one embodiment, the emitter is opposite the detector, that is, on opposite sides of the user's body part, for example, the user's finger. In another embodiment, the sensor is on the same side of the user's body and the sensor detects the reflected light. Yamakoshi explains “the light from the light source is separated by the active spectroscope and then irradiated to the living body [ ]. However, the transmitted light or reflected light may be separated for spectrum analysis after the light from the light source is irradiated to the living body. For example, the light can be separated by an array of plural light detectors [ ], each having a sensitivity only for specific wavelengths λ.”

The Yamakoshi processor uses a multi-regression analysis to extract “corresponding a known blood glucose concentration of the whole blood sample [ ] to an objective variable y, applying spectrum data of the whole blood sample [ ] to an explanatory variable x and deciding a multi-regression analysis blood glucose concentration computing model.”

A similar highly complex blood glucose measurement system is disclosed in U.S. Pat. No. 11,826,144 to Lee. That patent specification discloses, in one embodiment, a watch-based blood glucose sensor. In Lee's abstract, the blood glucose sensor is discussed as having a spectrometer “configured to emit a light to a skin of a user, detect the light returned from the skin, and measure a skin spectrum of the user from the detected light and a processor configured to estimate a biological substance in the user.

One embodiment in Lee '144 uses a blood sample solution which is subject to the spectral light analysis to obtain the baseline for the “watch-based” BG data acquisition sensor. Lee explains “When the skin spectrum for estimating the biological substance is measured by the measuring unit [ ], the processor [ ] may estimate the biological substance in the user on the basis of the measured skin spectrum and the unit spectrum. For example, when a skin spectrum for estimating blood sugar is acquired, the processor [ ] may calculate a predicted blood sugar value using [an] Equation [ ] below. Referring to Equation [ ], when the skin spectrum Sm for estimating the blood sugar is acquired, the processor [ ] may remove noise caused by a skin component by subtracting a background spectrum Sb from the skin spectrum Sm in order to increase a signal-to-noise ratio (SNR). Here, the background spectrum Sb may be a spectrum that is repetitively measured in reference state (e.g., a fasting state) at certain intervals for a certain time.”

The Lee '144 Equation estimates a blood sugar value by the ratio of BS to Su (BS divided by Su), wherein BS is the difference between Sm and Sb (Sm−Sb) and BG is the ratio of BS to Su plus RG (BS/Su+RG). RG is “a reference blood sugar value [ ] to the calculated estimated blood sugar value.” Lee explains “Here, the unit spectrum may be acquired using a biological-tissue simulation solution by the unit spectrum acquisition apparatus [ ] that has been described above and may be input in advance to the biological substance estimation apparatus [ ].”

The Predetermined Narrow Band within the 1350-1800 nm Sensory Band. The present wearable device BGD is different than the systems in Lee, Yamakoshi and the Ogawa/Yamakoshi article because (a) the present invention uses a predetermined NIR narrow band within the sensory 1350-1800 nm bandwidth and (b) the present invention does not massively process a returning or reflected NIR light spectrum. The present system generates NIR in a narrow transmission band within the NIR sensory bandwidth range (1350-1800 nm). A narrow NIR band within the NIR sensory bandwidth range is useful because the SIF value changes within the breadth of the NIR sensory bandwidth range (1350-1800 nm).

The Ogawa/Yamakoshi article refers to an application of “differential spectra” of NIR light to detect BG and then uses a Fourier transform near-infrared (FT-NIR) spectrometer for absorbance measurements. Yamakoshi '983 uses an “instantaneous spectrum analyzer [ ] to analyze the light transmitted through the living body or reflected thereon at different times.” The Lee '144 system uses a spectrometer and an IR transmitter which generates NIR and MIR light. The “unit spectrum may be acquired in the similar manner to the biological tissue.” The unit spectrum (the Lee '144 sensor unit) acquires light scattered and diffracted from a scatterer and a reflector in the unit spectrum. The “acquired spectra [includes] Spa and Sgs using a reference spectrum that is measured using a serum solution in a standard state.” The unit spectrum sensor system includes a spectrometer and reflector. Lee '144 explains “the measuring unit [ ] may use infrared spectroscopy or Raman spectroscopy . . . the measuring unit [ ] may measure the spectrum by utilizing various spectroscopies.”

The present invention is much simpler than the systems disclosed in the Lee '144 and Yamakoshi '983 disclosures and the Ogawa/Yamakoshi article because the present invention uses a differentiation or correlation process and then adjusts the signal levels based upon SIF values at the generated predetermined narrow-band NIR light.

Spectral Reflectance of Skin (FIGS. 1A and 1B). The present invention also accounts for the spectral reflectance of the skin. The use of NIR light to detect a user's blood glucose level is effected by the spectral reflectance of the skin. The NIST article titled “Reflectance Measurements of Human Skin,” and the NIST article titled “Spectral Reflectance Variability of Skin and Attributing Factors” by C. Cooksey, shows the same graph for reflectance of skin illuminated by NIR and other light wavelengths. The articles report that the key contributors to the spectral structure of human skin include water, melanin, and hemoglobin.

The Cooksey article states that for FIG. 1A (replicating a figure in Cooksey), “The reflectance spectrum of the representative of the mean (dashed line) with grey shaded area representing the population variability for all subjects. Representative spectra showing the range of variation in reflectance factors in the visible region (grey [shaded]) are also depicted.” In FIG. 1A, solid dashed line is the mean, the upper solid line and lower dashed-dot-dashed line depict the range of variation in the reflectance factors and the cross-hatched shaded area represents the population variability. Cooksey reports that overall variability in the spectral response is caused by significant concentrations of melanin.

Variability of Skin Reflectivity in Sensory Range. FIG. 1B replicates a portion of a graph in the Cooksey article specifically identifying that at 1450 nm, the skin reflectivity is at a low of about 5%. Also, FIG. 1B establishes that the skin reflectivity in the NIR sensory range (1350-1800 nm) of the present invention ranges from a high of 20% at 1350 nm to a low of about 5% at 1800 nm. At 1350 nm, the skin reflectivity is about 28%; at 1450 nm, the skin reflectivity is about 5%; at 1550 nm, the skin reflectivity is about 10%; at 1650 nm, the skin reflectivity is about 12%; and at 1800 nm, the skin reflectivity is about 10%.

The Cooksey study measures human skin reflectance on the inside of the participant's forearm. The variability of the reflectance spectra for a number of subjects measured was determined using statistical methods. “Although the reflectance spectra of human skin is the result of complex absorption and scattering events, it is typically described in terms of the absorption of melanin, oxy- and deoxygenated hemoglobin, and water.” The penetration depth increases as wavelength increases, reaching a maximum depth of 3.5 mm near 1090 nm. The test area was a large 2.54 cm diameter circle located on the inside of the subject's right forearm. The data in the Cooksey study was obtained by “integrating sphere-based spectrophotometers or spectrometers” to measure the reflectance of skin in vivo. Cooksey further reports “that the overall variability of skin reflectance can primarily be attributed to biological variability among individuals.” The Cooksey “spectrophotometer was used to acquire the 8°/h spectral reflectance factor of the subject's skin within the test area over the spectral range of 250 nm to 2500 nm at a wavelength interval of 3 nm . . . [and the spectral] spread is particularly tight at 1450 nm, although it should be noted that overall uncertainty is comparable to the observed reflectance factors at this wavelength.”

Infiltration of NIR. It should be noted that Cooksey indicates an infiltration of 3.5 mm at 1090 nm in the user's inside forearm whereas Ahmed's article used NIR in the range of 740-940 nm and indicated an infiltrate of 5 mm into the skin tissue. However, Cooksey used a large 2.54 cm (that is 254 mm) circular test area.

In contrast to the Cooksey system, the present invention does not use integrating sphere-based spectrometers to measure the reflectance of skin and blood glucose in vivo. The present invention uses a differential process which is pre-loaded with the conventionally acquired BG data matched with BG sensed data wherein the BG sensed data is generally acquired at the same time as the conventionally acquired BG data. The differential resultant is further adjusted for subcutaneous interference factor (SIF) related to the narrow NIR band generated and penetrating the user's skin.

Subcutaneous Interference Factors (SIF). The following SIF Adjustment table provides examples of subcutaneous interference factor (SIF) values which shows, given a particular narrow band wavelength of the NIR transmitted/returned signal, 30% of the signal represents glucose in the blood, and that returned signal needs to be increased to account for the 4% water absorption, and then decreased by 20% to account for the reflectivity of NIR in the skin. The resultant SIF means that, at 1350 nm, 14% of the sensed signal represents glucose in the blood. This is the SIF adjusted, contributory glucose percent value of the then-detected signal. At initialization at that 1350 nm wavelength, the baseline blood glucose signal is modified such that 14% of the signal represents the reflected NIR light sensed by the NIR receiver/sensor. During post-initialization or normal operations, when operating on then-acquired NIR sensed signals at 1350 nm, 14% of the signal represents blood glucose and that 14% adjusted signal is compared to the baseline BG signal level. The resultant (the differential between the then-acquired signal and the baseline signal) provides an indication or a representation of the user's then-current blood glucose level.

The SIF adjusted, contributory glucose percent value of the then-detected signal is correlated to the generated narrow NIR band centered on the centerpoints or peak NIR radiation for example, at 1350, 1450, 1550, 1650, and 1880 nm wavelengths. These are examples of the SIF adjusted, contributory glucose percent values at those narrow NIR bands. Other narrow band width may be used such as 1375, 1600 or 1735 nm. Since the reflectivity of water is nil over the NIR sensory range, this factor is not accounted for in the SIF contributory adjustment. The invention establishes that given the % of Glucose Reflectivity in the designated narrow NIR band plus the % of Absorption Spectrum of Water and less than the % Reflectivity of Skin generates the SIF Contributory Adjustment Value. The table below shows several narrow NIR bandwidths, at designated centerpoint wavelengths, and the resultant SIF contributed glucose % at that designated narrow NIR band. Other designated centerpoint wavelengths (for example, 1400 nm, 1500 nm, 1600 nm, etc.) may be used. The SIF contributory adjustment % changes for those other transmitted/returned narrow bands. Further, if different studies show different values % of Glucose Reflectivity, % of Absorption Spectrum of Water, and/or % Reflectivity of Skin, the SIF contributory values set forth below many changes. A statistical combination of these different studies will improve the valuation of SIF.

TABLE
SIF Adjustment

| | Absorption | | | SIF |
|---|---|---|---|---|
| NIR Band - | Spectrum | Reflectivity | Reflectivity | Adjusted |
| centerpoint | H2O % | of Skin % | of Glucose % | Value % |
| 1350 | +4 | −20 | +30 | 14 |
| 1450 | +4 | −5 | +42 | 41 |
| 1550 | +5 | −10 | +70 | 65 |
| 1650 | +15 | −12 | +68 | 71 |
| 1800 | 0 | −10 | +55 | 45 |

The SIF contributory adjustment, as a percentage, can be applied in several mathematical manners to the sensed data at (a) initialization and (b) post-initialization during normal operations.

In should be noted that the narrow transmission band centerpoints in the SIF Table can be any NIR light wavelength as long as it falls within the NIR sensory bandwidth 1350-1800 nm. NIR transmission light generators/transmitters are sold listing the NIR wavelength centerpoint. The same is true for NIR receiver sensors. However, currently commercially available NIR transmitters generate light over a small bandwidth centered on and about their designated wavelength centerpoint, that is the highest light intensity level. Commercially available NIR light transmitters (circa 2024) are sold with product specifications indicting both the peak intensity wavelength and a spectral line half width wavelength Δλ (delta wavelength) for that same NIR transmitter. The narrow NIR band includes the spectral line half width wavelength notwithstanding the fact that, for 1350 nm, the lower spectral line half width wavelength falls outside the NIR sensory range of 1350-1800 nm.

As a result of this wavelength spread for currently available NIR transmitters, that is the spectral line half width wavelength Δλ, the “NIR sensory bandwidth of 1350-1800 nm” encompasses these half width wavelengths such that the sensory bandwidth includes (a) at NIR light transmission at peak centerpoint 1350 nm, a spectral line half width wavelength Δλ no less than 1300 nm and (b) at NIR light transmission at centerpoint 1800 nm, a spectral line half width wavelength Δλ no more than 1850 nm. Stated otherwise, the “NIR sensory bandwidth of 1350-1800 nm” is measured at the centerpoint or high intensity NIR light transmission peak of the NIR transmitters. The NIR receivers or sensors may also have reception bandwidths for centerpoint/peak reception and spectral line half width wavelength Δλ reception.

For example, GMKJ a/k/a Shenzhen Guangmai Electronics Co., Ltd., of Shenzhen, China, makes Far Infrared LED 1350 nm (LEDs are available at peak wavelengths 1350 nm and 3535 nm). NIR transmitters are sold with specifications for peak emission wavelength λp data for a particular NIR transmitter (for example, a 1350 nm NIR transmitter) and spectral line half width wavelength Δλ data for that same NIR transmitter. See, for example, data from Marktech Optoelectronics of Mansfield, TX and CCS Inc., member of the Optex Group, located in Boston, MA. CCS sells 1300 nm, 1450 nm, 1550 nm, 1650 nm LED transmitters and the 1300 nm LED has a radiating bandwidth range of 1250-1325 nm at 50% radiant intensity, the half width wavelength Δλ. The approximate SIF contributory adjustment value at 1300 nm for that CCS LED is nearly 0% because the skin reflectivity (35% (higher than % at 1350 nm)) and the water absorption contributory factor (4%) plus the glucose reflectivity (30% (lower than % at 1350 nm)) is nearly cancels out the 35% skin reflectivity contributory factor.

The discussion of currently available NIR transmitters is not meant to establish accurate SIF adjustment values but rather to show how a person of ordinary skill in the art may use the information in this application and the commercial LED data (circa 2024) to select the NIR narrow transmission band for a quasi-medical grade NIR transmitter, match that transmitter to the NIR receiver, and establish the SIF value for a BG device given the SIF values at certain wavelengths within the NIR sensory range and the current state of the art of NIR transmitters and sensors. Other factors such as power consumption and heat generation and period of illumination or light transmission have not been considered in the foregoing discussion of the GMKL, Marktech and CCS products. Stated otherwise, those products may be completely non-functional for the user-friendly quasi-medical grade BG device disclosed herein. The BGD is quasi-medical because the BG device is not meant to replicate the conventionally determined blood glucose level. However, the BG device is meant to be user-friendly and to quickly estimate the user's blood glucose level. Also, if more accurate SIF data can be gathered from future studies and if more precise NIR transmitters and sensors are available, the present invention may qualify as a medical grade BG detection system.

Structural and Functional Details of the Invention. The following describes the structural and functional aspects of embodiments of the invention.

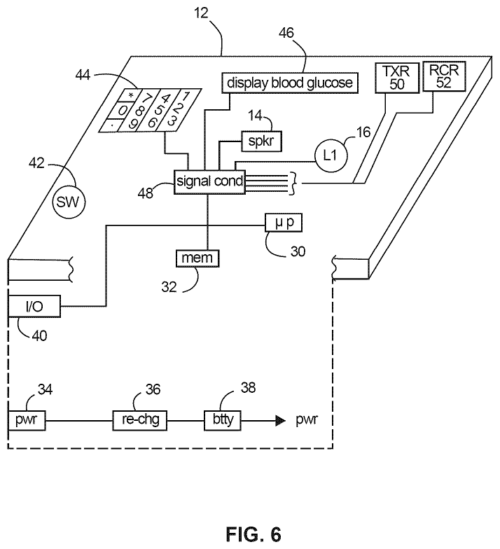

FIG. 3 diagrammatically illustrates one embodiment of the wearable device for measuring blood glucose levels based on glucose reflectivity configured and embodied in a watch-like structure 110. The BGD watch 110 has a watch body 114 and user-facing face 120 with various user interfaces (UIs) as described later. The NIR transmitter and NIR sensor (or combinatory transmitter/sensor) is mounted in the watch 110 on an opposite side (the skin-side) of watch face 120. The operations which may be associated with the user actuatable button or stem 122 are discussed below. Band element 112 and adapted to the attachable coupled to opposing band element 116. The BGD housing 12 in FIG. 6 is encased in the watch body 110.

FIG. 4 diagrammatically illustrates another embodiment of the BGD wearable device configured and embodied as a patch-like structure 150. The patch 150 includes a transmitter-sensor-processor body 152 in a central location in the patch. The thin, rectangular body represents the housing 12 in FIG. 6. The patch, in one embodiment, is attachable to the skin of the user by removal of non-stick panel segments 153, 154, by the user pealing back further non-stick elements 161, 162, thereby exposing the underlying adhesive surface (not shown) adapted to the attachable and detachable on the skin of a user. The BGD patch is attached at a location on the user's body within the infiltration range of arterial blood. The NIR transmitter/sensor is located on surface 151. The UI panel (keypad-display module) is located on the opposite face surface 151. The shape and size of the patch 150 can be altered as needed, that is, it is not necessary that the patch is rectangular, square, or other-shaped.

FIG. 5A diagrammatically illustrates another embodiment of the BGD wearable device configured and embodied as a medallion 170. A lanyard 172, in one embodiment, is long enough to hang medallion 170 about the neck of a user. The medallion 170 has a face 174 for the user interface or UI which is described in detail later. The NIR transmitter and NIR sensor (or combinatory transmitter/sensor) is mounted in medallion 170 on an opposite side of medallion face 174. User actuatable buttons or other UI hardware items may be disposed on the medallion side 176 or on face 174. Medallion face 174 carries the UI elements (keypad, display with or without audio speakers. The shape and size of medallion 170 can be altered as needed, that is, it is not necessary that the medallion be round, square, or rectangular. In a combinatory medallion/patch system, the medallion may have small adhesive pads permitting the user to temporarily attach the medallion to his or her skin to obtain BG signals.

FIGS. 5B and 5C diagrammatically illustrate eyeglasses embodiments of the BGD wearable device (FIG. 5B illustrating a nose bridge mounted or embodied BGD and FIG. 5C illustrating a temple mounted or embodied BGD). FIGS. 5D and 5E show an earring-based BGD and a stud-based BDG. These BGD systems all use a smart phone (see FIG. 5B, phone 310) are generally referred to as body-mountable structure. The BGD is an onboard unit which interacts with: for the eyeglass temple (FIG. 5B), a temple-ear skin-side surface; for the eyeglass nose bridge (FIG. 5C), a nose bridge skin-side surface; for the clip-on earring (FIG. 5D), a clip earring ear skin-side surface; for the stud mounted earring (FIG. 5E), a stud earring ear skin-side surface; and for the stud mounted nose body (also FIG. 5E), a nose skin-side surface.

Similar numerals refer to similar items in all the drawings. Eyeglasses 210 either embody or carry a BG device 300 in the nose bridge of the eyeglasses as shown in FIG. 5B or on the glasses temple 214 as shown in FIG. 5C. The BGD 300 wirelessly communicate with a smart phone 310 (diagrammatically illustrated in FIG. 5B) as shown by double-headed arrows 305. The hardware and software operations of the BGDs in FIGS. 5B, 5C, 5D, and 5E are discussed later in connection with FIG. 6. It should that the BGD NIR transmitters and sensors are disposed on the skin-side of BGD 300. Hence, in FIG. 5B, the NIR transceivers are adjacent the skin of the user's nose. In the temple mounted version of FIG. 5C, the BGD transceivers are on an inboard temple position facing the ear of the user. Once the user engages the initialization mode of the BGD, as explained later, the BGD may capture infiltration data such that, at a later time, post-initialization, when the user wishes to detect his or her blood glucose level (or a representation of that BG level), the user would reposition his or her eyeglasses to the initialization position. As explained later, the BGD may have detection and alarm systems to indicate to the user that the eyeglasses have been returned to the initialization position. Smart phone 310, operating the BG APP, issues alarms and ultimately displays to the user his or her BG level or a representation thereof.

FIG. 5D diagrammatically illustrates a clip-on earring embodiment of the BG device which operates in conjunction with the smart phone APP sub-system shown in FIG. 5B. The wireless communication to the phone is shown by arrows 305. The clip-on earring has an earring body 230 which is removably amounted to the user's ear by a clip mechanism 231. The earring body has a skin-side surface 232. The BGD NIR transceiver (that is, the transmitter and the receiver units or unit) can be disposed anywhere on skin-side surface 232 or and the inboard protruding pad 234 opposite clip mechanism 231. FIG. 5D shows the BGD housing 300 on the clip-facing side of the earring 230. Similar to the eyeglasses BGD embodiment, the clip-on earring embodiment may include a positioning and infiltration testing function thereby enabling the user to reposition the earring at the initialization position.

FIG. 5E diagrammatically illustrates a stud-mounted earring or a stud-mounted nose BG device 250 which operates in conjunction with the smart phone APP sub-system shown in FIG. 5B. The wireless communication to the phone is shown by arrows 305. In either the stud-mounted earring or the stud-mounted nose body, the BGD housing, or a portion of the BGD housing, is mounted in structure 250 of the earring/nose body. The BGD housing and encapsulated circuitry 300 communicates wirelessly with the smart phone as noted by wireless connection 305. The earring/nose body 250 has a skin-side surface 256. The BGD NIR transceiver (that is, the transmitter and the receiver units or unit) can be disposed anywhere on skin-side surface 256. The stud-based earring/nose body 250 is removably amounted to the user's ear or to the user's nose by stem 252 which protrudes through the respective body part. An earring back or a nose stud back, not shown, is placed on stem 252 by having back threaded onto terminal stem end 254. The back has an aperture into which stem 252 passes. The back may be threaded, screwed or detent coupled to the stem. Once the back is mounted on stem 252, the skin-side surface 256 of the earring/nose body is adjacent and next to the ear of the user or the nose of the user. In either case, the BGD NIR transceiver (that is, the transmitter and the receiver units or unit) can be disposed anywhere on skin-side surface 256. Similar to the eyeglasses BGD embodiment, the studded nose/earring embodiment in FIG. 5E may include a positioning and infiltration testing function thereby enabling the user to reposition the earring at the initialization position.

FIG. 6 diagrammatically illustrates, in block diagram form, the blood glucose detector and electrical components associated therewith. It should be noted that many, if not all, of the electronic components discussed herein may be configured on one or more ICs. The components in FIG. 6 are effectively described functionally to sense, record, process, and display or announce to the user his or her then-current BG level. FIG. 6 diagrammatically illustrates, in block diagram form, the functional major components of the blood glucose detector. Housing 12 includes on/off switch 42, keypad 44 and display 46. In another embodiment, mechanical user interfaces (switch 42) and the keypad 44 may be replaced by a touchscreen such as used on smart phones circa 2024. Also, in another embodiment, voice recognition and audio input and output may replace the UI elements discussed herein. In other words, regarding the BG outputs alerting the user as to his or her BG level, the audio-based BGD would audibly announce the BG level to the user.

Returning to the embodiment illustrated in FIG. 6, preferably, keypad 44 (which may be a combinatory display/keypad module) has indicia 1-9, 0, an asterisk and a period. Alternatively, or in addition thereto, keypad 44 may include alphabetic character groups which are sometimes found on the touch keypad of a smart phone. Keypad UI may include other non-numeric keys. The UI also includes BG display 46 which visually presents the BG data to the user. In an enhanced embodiment, the display 46, in conjunction with processor 30 and memory 32 may present instructions to the user how to upload conventionally obtained BG data, set up the initialization process, and other UI operational instructions. Further, the UI may include a speaker 14 for as an audio interface. A microphone may be added to the UI.

IR transmitter (TXR) 50 is located at one surface of housing 12 and IR receiver (RCR) 52 is co-located on the housing surface with the transmitter 50. Both are located on the skin-side of the BGD. Typically, the user interfaces or UIs are located on the front face of the BGD, opposite the transmitter/sensor skin-side. In some embodiment, the transmitter and receiver may be combined into one unit. A positional light emitter L1, alerting the user as to the proper position of the BGD on the user's skin, wrist, arm, etc., may be part of the user's interface or UI.

To assure re-positional accuracy (matching the initialization position of the BGD to the then-proposed-BG sensing position), during initialization, the BGD may acquire infiltration data via the NIR transmitter/sensor system and, during re-positioning of the BGD to obtain then-current BG levels, the processor and memory may seek to match the earlier acquired infiltration data with then then-acquired infiltration data and audibly or visually (light L1 or display 46) or both announce that the then-present sensor position aligns with the earlier-acquired infiltration position of the BGD. If the infiltration data does not match, a no-match alarm (audio or visual or both) is provided to the user.

For example, the infiltration data may be an infiltration factor (IF) which is derived from the underlying data in the SIF calculations, after accounting for the % absorption of water and removing the % glucose reflectivity value. For example, when the NIR transmitter has a centerpoint or peak intensity at 1350 nm, the absorption spectrum of water is 4%, and the reflectivity of skin 20%, therefore the IF value is 24% of the returned, sensed NIR light signal because the water absorbed 4% of the NIR light and reflectivity of skin reflected 20% of the NIR light signal. This IF contributory adjustment ignores the glucose data present in the returned, sensed NIR signal.

This IF contributory adjustment value, taken at the time the baseline blood glucose level is determined (upon upload of the conventionally acquired BG level data), is the sum of the % absorption spectrum of water (at 1350 nm, this is 4%, indicating that 4% of the sensed signal is lost to absorption), and the % reflectivity of skin (at 1350 nm, this is 20%). It is believed that this IF contributory adjustment is the same at initialization and post-initialization data capture.

Other IF detection methods may be used to confirm re-positioning of the BGD on the user's skin.

The BGD is controlled by a processor, preferably microprocessor (pp) 30, operating in conjunction with memory (mem) 32. These elements may be combined on a single IC. Power is supplied to the system via a power (pwr) port 34, a recharging (re-chg) subsystem 36 and a battery (btty) 38. This power transmission system may be replaced or supplemented by the “touch-based” power recharge systems used on some smart phones circa 2024. Preferably, the BGD is a digital device with which accepts digital information and outputs digital information via input/output I/O port 40. In some embodiments, I/O port may include a near field receiver/transmitter, such a Bluetooth enabled, such that the BGD is communicatively coupled to another device, such as a smart phone operating an APP and paired with the BGD via Bluetooth or other near filed communications link. Also, during initialization, the BGD may be wired or wirelessly communicatively coupled to the computer-based system obtaining the conventionally obtained BG data. Processor 30, memory 32, port 40 and the other electronic BGD components are connected to each other via bus or circuitry connections. One or more signal conditioners 48 convert any signals generated by the microprocessor into signals compatible to drive the UI elements, that is, keypad 44 and BG level display monitor 46 and to drive speaker (spkr) 14 and positional indicator light 16 (L1). Conditioners 48 many also be on an IC with the processor and memory. Display 46 shows the user-understood displayed blood glucose number, level or range indicator to the user. Further, signal conditioners 48 are coupled to IR transmitter 50, IR receiver 52 and other components as needed.

In the body-mounted BGD systems, such as the eyeglasses in FIGS. 5B and 5C, the clip-on earring in FIG. 5D and the studded earring in FIG. 5E (also the studded nose body in FIG. 5E), portions of the BGD system diagrammatically illustrated in FIG. 6 are placed in the onboard, skin-contacting BGD bodies 300 while other portions of the BGD system use functional aspects of the smart phone which is communicatively linked to the BGD body during (i) the initialization process and (ii) the post-initialization or normal operational. As noted earlier, many of the BGD electronic components may be configured on one or more ICs. The onboard, body-mounted BGD components should (a) generate the NIR light, (b) sense the returning NIR light signal, (c) enable communications with the smart phone APP system, and have some onboard power source (such as a watch battery). The onboard body also includes signal conditioners 48 and chip-configured processor and memory for operating the NIR light, the NIR light sensor, and communicating with the smart phone. The onboard battery also needs a power converter for the NIR light, sensor and digital processor and memory. In an advanced system, the onboard, body-mounted BGD has a battery and a recharging portal for recharging the onboard battery. See FIG. 6 and the functionality of power port 34, recharger 36 and battery 38. The body-mounted BGD is a digital device which accepts digital information and outputs digital information via a wireless input/output I/O port 40. The onboard structure's I/O port 40 includes a near field receiver/transmitter, such a Bluetooth enabled, such that the BGD is communicatively coupled to the smart phone 310 operating the APP and paired with the BGD via Bluetooth or other near filed communications link.

In the body-mounted BGD systems (the eyeglasses, the clip-on earring, the studded earring, and studded nose body), all the UIs are located and operated on by the smart phone display-touchscreen system, the cell phone processor and the cell phone memory. The body-mounted BGD systems may have a small UI depressible switch (not shown) to enable wireless coupling to the smart phone communications portal.

The smart phone APP replicates the functions discussed above including (1) an on/off system master control (typically the ON control is activated by depressing the APP icon on the smart phone and the OFF control, replicating the function of switch 42 in FIG. 6, is activated by closing the APP on the phone), (2) keypad 44 with the cell phone touch screen functionality, and (3) the display 46. The smart phone may enable an UI with voice recognition and audio inputs and outputs. The APP and phone replicate presentation of the user's BG level and re-positional alarm and acceptance indicators (acceptance matching the position at initialization).

Similar to the BGD in housing 12, the smart phone-based UI also includes functions replicating BG display 46 which visually presents the BG data to the user, provides processing functions similar to processor 30 and data storage similar to memory 32. The phone-enabled embodiment may present instructions to the user how to upload conventionally obtained BG data, set up the initialization process, and other UI operational instructions. Further, the cell phone system UI includes a function similar to speaker 14 for an audio interface. The phone also mimics the microphone function. Smart phone-enabled BGDs may have, on the phone-side, functional equivalents of positional light emitter L1, alerting the user as to the proper position of the BGD on the user's skin.

As described earlier, in the body-mounted BGD, IR transmitter (TXR) 50 is located on the skin-side surfaces of the eyeglasses, the clip-on earring and the studded earring and studded nose body. The IR receiver (RCR) $2 is co-located on the side-side surface.

Initialization Process: The following is a summary of one initialization process for the BGD. a. Start-up BGD (assumes power circuit is ON); b. User inputs initialization start code (e.g., “000”); c. User or User's healthcare professional gathers conventionally obtained BG data; d. User places BGD device on a skin region that he or she will use in the future to obtain then-current BG data; e. Positional light L1 blinks to confirm location data (optional audible confirm); skin location/infiltration data stored in memory; f. User or User's healthcare professional inputs conventionally obtained BG data (input may be entered manually vis keypad or wired linkage to I/O 40, or wirelessly via near field RF, such as Bluetooth (BGD needs to be paired with compatible data upload device, such as a smart phone with an APP communicatively interacting with the BGD or other computer-based unit); g. L1 full ON to show conventional BG data upload complete (optional display 46 indicates CONVENTIONAL UPLOAD OK and/or audio approval provided); h. Processor activates the NIR transmitter and receiver to gather the user's then-current sensed BG data (this BGD operation should be at or near the time the conventional BG data is obtained); i. Processer normalizes the user's then-current sensed BG data with the conventional uploaded BG data. Processor labels the correlated data as the BG baseline data (then data stored in memory); j. Light L1 periodically flashes indicating BG baseline data stored. Optional audio/display 46 announcement/presentment; and k. BGD system active and operational for future acquisition of BG data.

Operational Process (Post Initialization): The following is a summary of one operational process for the BGD. a. Start-up or wakeup BGD via one or more of the UI modules; b. User places BGD device on a predetermined skin region (earlier identified at initialization; if a watch-based BGD, the initialization is processed with the watch on the user's wrist—during the operational phase, the watch is generally at the same location; some BGDs may require the skin-side of the BGD watch to be on the anterior of the wrist during initialization and during post-initialization operations); c. Processor confirms YES current location matches earlier stored location data or, if NO, BGD not on predetermined skin region (infiltration data may be used); d. Location match-YES: light L1 blinks to confirm location data (optional audible confirm); e. Location match-NO: light L1 remains OFF, audio indicator may announce REPOSITION (alternatively, display may present REPOSITION alarm). If NO, repeat earlier operational process steps; f. Light L1 full ON to show ready to acquire BG data; g. Processor activates the NIR transmitter and receiver/sensor to gather the user's then-current sensed BG data; h. Light L1 full ON to show then-current BG data obtained (optional, store raw BG data in memory with date/time); i. Processer compares then-current BG data with the BG baseline data and obtains resultant differential (store Resultant in memory with date/time); j. modify the then-acquired data with the subcutaneous interference factor (SIF) value. k. Convert Modified Resultant to a User-friendly display presentation (simple numeric; complex numeric level/range; simple visual GOOD/green, OK/blue, or BAD/red); l. Light L1 periodically flashes indicating then-current BG data stored. Optional audio announcement; and m. Optional: periodic upload raw BG data, date/time of raw data acquisition, and BG Modified Resultant data via wired or wireless communicative coupling to an external complementary device, such as a smart phone-wireless connection with a phone-mounted APP or wireless to other computer-based system(s).

In other embodiments, the keypad may be replaced by a touch screen display which, during initialization, shows a keypad to enable the user to input baseline BG data into the BGD. In this manner BG display panel 46 is replaced by the touch screen which also acts as a BG level display monitor. BG display 46 receives a BG displayable signal produced by the processor and the memory. Also, the processor converts the then-detected BG signal into a corresponding BG displayable signal (a data conversion routine) such that the user can compare the then-detected BG signal to the doctor recommended BG level.

Location positional detection process. As for the location detection process, the memory stores, at the time of the initialization stage, infiltration data (at a time of obtaining the baseline blood glucose level) accounting for, at the NIR light narrow transmission band, the absorption spectrum of water and the reflectivity of skin. At the time of acquiring the then-detected blood glucose signal, the processor obtains a then-detected infiltration datapoint which accounts for the absorption spectrum of water and the reflectivity of skin at the NIR light narrow transmission band. The processor then compares the stored infiltration data to then-processed infiltration datapoint, and issues an alarm if the differential therebetween exceeds a predetermined positional value. The match-YES positional threshold may be wide to accommodate variations in the user's blood flow.

The present invention could be produced in hardware or software, or in a combination of hardware and software, and these implementations would be known to one of ordinary skill in the art. The system, or method, according to the inventive principles as disclosed in connection with the preferred embodiments, may be produced on a single computer IC chip having separate elements or means for performing the individual functions or steps described or claimed or one or more elements or means combining the performance of any of the functions or steps disclosed or claimed, or may be separate IC components interconnected by any suitable means.

According to the inventive principles as disclosed in connection with the embodiments, the invention and the inventive principles are not limited to any particular kind of computer chip system but may be used with any general purpose processor, as would be known to a person of ordinary skill in the art, arranged to perform the functions described and the method steps described herein. The operations of such a processor and memory, as described above, may be according to a computer program contained on a medium for use in the operation or control of the processor, as would be known to person of ordinary skill in the art. The computer medium which may be used to hold or contain the computer program product, may be a fixture of the computer such as an embedded memory or may be on a transportable medium such as a disk, as would be known to one of ordinary skill in the art.

The invention is not limited to any particular computer program or logic or language, or instruction but may be practiced with any such suitable program, logic or language, or instructions as would be known to one of ordinary skill in the art. Without limiting the principles of the disclosed invention any such computing system can include, inter alia, at least a computer readable medium allowing a processor to read data, instructions, messages or message packets, and other computer readable information from the computer readable medium. The computer readable medium may include non-volatile memory, such as ROM, flash memory, or other optical memory storage devices, and other permanent storage. Additionally, a computer readable medium may include, for example, volatile storage such as RAM, buffers, cache memory, and network circuits.

Furthermore, the computer readable medium may include computer readable information in a transitory state medium such as a network link and/or a network interface, including a wired network or a wireless network, that allow a computer to read such computer readable information.

The claims appended hereto are meant to cover modifications and changes within the scope and spirit of the present invention.

What is claimed is:

1. A blood glucose detector adapted to be placed on a user's wrist or other skin surface at a near infrared (NIR) light infiltration location such that arterial blood flow is detected at the wrist or other skin surface by the NIR light, and the blood glucose detector used in conjunction with an earlier obtained baseline blood glucose data set representing an earlier conventionally obtained blood glucose data comprising:
a user-wearable housing for a processor, a processor-coupled memory, a NIR transmitter and a NIR sensor, the memory storing therein the baseline blood glucose data set, the user-wearable housing and the NIR transmitter adapted to be disposed on the infiltration location of the user's wrist or other skin surface in a detection mode;
in a detection mode, the NIR transmitter generating NIR light in a predetermined narrow transmission band which narrow band is within a NIR sensory bandwidth of 1350-1800 nm;
the NIR sensor detecting reflected light from arterial blood glucose representing a then-detected blood glucose signal;
the memory storing a predetermined subcutaneous interference factor (SIF) contributory value for the NIR light narrow transmission band;
the processor correlating the detected blood glucose signal with the baseline blood glucose data set as modified by the SIF value and generating a displayable blood glucose level for the user; and
the housing having a user interface display, the display coupled to the processor and displaying, during the detection mode, the displayable blood glucose level to the user enabling a user comparison with a healthcare recommended blood glucose level.

2. The blood glucose detector as claimed in claim 1 wherein the predetermined SIF value, stored in the memory for the narrow transmission band, accounts for a predetermined glucose reflectivity, a predetermined absorption spectrum of water, and a predetermined reflectivity of skin.

3. The blood glucose detector as claimed in claim 2 wherein the predetermined SIF value is an amalgamation of the predetermined glucose reflectivity, the predetermined absorption spectrum of water, and the predetermined reflectivity of skin.

4. The blood glucose detector as claimed in claim 3 wherein the predetermined SIF value includes a reduction accounting for the predetermined reflectivity of skin.

5. The blood glucose detector as claimed in claim 1 wherein the NIR sensory bandwidth of 1350-1800 nm encompasses (a) at a NIR light transmission centerpoint 1350 nm, a spectral line half width wavelength Δλ no less than 1300 nm, and (b) at a NIR light transmission at centerpoint 1800 nm, a spectral line half width wavelength Δλ no more than 1850 nm.

6. The blood glucose detector as claimed in claim 4 wherein the NIR sensory bandwidth of 1350-1800 nm encompasses (a) at a NIR light transmission centerpoint 1350 nm, a spectral line half width wavelength Δλ no less than 1300 nm and (b) at a NIR light transmission at centerpoint 1800 nm, a spectral line half width wavelength Δλ no more than 1850 nm.

7. The blood glucose detector as claimed in claim 4 including storing in the memory an infiltration data at a time of obtaining the baseline blood glucose level accounting for, at the NIR light narrow transmission band, the absorption spectrum of water and the reflectivity of skin, the processor storing the infiltration data in memory, and, at the time of acquiring the then-detected blood glucose signal obtaining a then-detected infiltration data, and comparing the stored infiltration data to the infiltration data, and issuing an alarm if the differential therebetween stored and detected infiltration data exceeds a predetermined positional value.

8. A method for detecting blood glucose on a user's wrist or other skin surface at a near infrared (NIR) light infiltration location such that arterial blood flow is detected at the wrist or other skin surface by the NIR light comprising:
during an initialization phase, obtaining a baseline blood glucose data set correlated to a then-conventionally obtained blood glucose data:
providing a user-wearable housing for a processor, a processor-coupled memory, a NIR transmitter and a NIR sensor, the NIR transmitter generating light in a predetermined narrow transmission band which narrow band is within a NIR sensory bandwidth of 1350-1800 nm;
storing in memory the baseline blood glucose data set;
in a post-initialization detection mode, placing the user-wearable housing and the NIR transmitter on the infiltration location of the user's wrist or other skin surface;
in the detection mode, transmitting NIR light into the infiltration location;
detecting, with the NIR sensor, reflected NIR light from arterial blood glucose representing a then-detected blood glucose signal;
prior to initialization and detection, storing in memory storing a predetermined subcutaneous interference factor (SIF) contributory value for the NIR light narrow transmission band;
correlating, with the processor, the detected blood glucose signal with the baseline blood glucose data set as modified by the SIF contributory value; and
generating a displayable blood glucose level for the user based upon the correlated and signals as modified by the SIF contributory value.

9. The method for detecting blood glucose as claimed in claim 1 wherein the predetermined SIF value accounts for a predetermined glucose reflectivity, a predetermined absorption spectrum of water, and a predetermined reflectivity of skin.

10. The method for detecting blood glucose as claimed in claim 9 wherein the predetermined SIF value is an amalgamation of the predetermined glucose reflectivity, the predetermined absorption spectrum of water, and the predetermined reflectivity of skin.

11. The method for detecting blood glucose as claimed in claim 10 wherein the predetermined SIF value includes a reduction accounting for the predetermined reflectivity of skin.

12. The method for detecting blood glucose as claimed in claim 8 wherein the NIR sensory bandwidth of 1350-1800 nm encompasses (a) at a NIR light transmission centerpoint 1350 nm, a spectral line half width wavelength Δλ no less than 1300 nm, and (b) at a NIR light transmission at centerpoint 1800 nm, a spectral line half width wavelength Δλ no more than 1850 nm.

13. The method for detecting blood glucose as claimed in claim 11 wherein the NIR sensory bandwidth of 1350-1800 nm encompasses (a) at a NIR light transmission centerpoint 1350 nm, a spectral line half width wavelength Δλ no less than 1300 nm and (b) at a NIR light transmission at centerpoint 1800 nm, a spectral line half width wavelength Δλ no more than 1850 nm.

14. The method for detecting blood glucose as claimed in claim 11 including storing in the memory an infiltration data at a time of obtaining the baseline blood glucose level accounting for, at the NIR light narrow transmission band, the absorption spectrum of water and the reflectivity of skin, and, at the time of acquiring the then-detected blood glucose signal, obtaining a then-detected infiltration data, comparing the stored infiltration data to the infiltration data, and issuing an alarm if the differential therebetween stored and detected infiltration data exceeds a predetermined positional value.

15. A blood glucose detection system including a body-mountable blood glucose detector and a communicatively linked smart phone, the body-mountable detector adapted to be placed on a user's ear skin surface or nose skin surface at a near infrared (NIR) light infiltration location such that arterial blood flow is detected at the user's ear skin surface or nose skin surface by the NIR light, the smart phone having a memory storing therein an earlier obtained baseline blood glucose data set representing an earlier conventionally obtained blood glucose data, the blood glucose detection system comprising:
the body-mountable detector including:
an onboard unit mounted on or in one body-mountable structure from a group of body-mountable structures including an eyeglass temple coupled to one eyeglass, an eyeglass nose bridge coupled to another eyeglass, a clip-on earring body, a stud mounted earring body, and a stud mounted nose body, a respective body-mountable structure having a corresponding skin-side surface from the group of skin-side surfaces consisting of: for the eyeglass temple, a temple-ear skin-side surface; for the eyeglass nose bridge, a nose bridge skin-side surface; for the clip-on earring, a clip earring ear skin-side surface; for the stud mounted earring, a stud earring ear skin-side surface; and for the stud mounted nose body, a nose skin-side surface;
a NIR transmitter and a NIR sensor encased in the onboard unit, the NIR transmitter and NIR sensor located on the corresponding skin-side surface of the respective body-mountable structure;
the onboard unit having control processor, coupled to an onboard power supply to initiate NIR light transmission via the NIR transmitter and enable sensing of the return NIR light via the NIR sensor and enable wireless communications via a wireless communications port with the smart phone, the NIR transmitter generating NIR light in a predetermined narrow transmission band which narrow band is within a NIR sensory bandwidth of 1350-1800 nm;
the NIR transmitter and NIR sensor, on the corresponding skin-side surface of the onboard unit's body-mountable structure, adapted to be disposed on the infiltration location of the user's ear skin surface or nose surface in a detection mode; and
the NIR sensor detecting reflected light from arterial blood glucose representing a then-detected blood glucose signal;
the control processor adapted to send the then-detected blood glucose signal to the communicatively coupled smart phone;
the smart phone having a processor coupled to the memory, at least a user interface display and a communications module, the communications module enabling wireless communications with the onboard unit;
the memory storing a predetermined subcutaneous interference factor (SIF) contributory value for the NIR light narrow transmission band;
the processor correlating the then-detected blood glucose signal with the baseline blood glucose data set as modified by the SIF value and generating a displayable blood glucose level for the user; and
the processor, during the detection mode, displaying a representative blood glucose level to the user enabling a user comparison with a healthcare recommended blood glucose level.

16. The blood glucose detection system as claimed in claim 15 wherein the predetermined SIF value, stored in the memory for the narrow transmission band, accounts for a predetermined glucose reflectivity, a predetermined absorption spectrum of water, and a predetermined reflectivity of skin.

17. The blood glucose detection system as claimed in claim 16 wherein the predetermined SIF value is an amalgamation of the predetermined glucose reflectivity, the predetermined absorption spectrum of water, and the predetermined reflectivity of skin.

18. The blood glucose detection system as claimed in claim 17 wherein the predetermined SIF value includes a reduction accounting for the predetermined reflectivity of skin.

19. The blood glucose detection system as claimed in claim 15 wherein the NIR sensory bandwidth of 1350-1800 nm encompasses (a) at a NIR light transmission centerpoint 1350 nm, a spectral line half width wavelength Δλ no less than 1300 nm, and (b) at a NIR light transmission at centerpoint 1800 nm, a spectral line half width wavelength Δλ no more than 1850 nm.

20. The blood glucose detection system as claimed in claim 18 wherein the smart phone includes a user interface alarm, the detection system including storing in the memory an infiltration data at a time of obtaining the baseline blood glucose level accounting for, at the NIR light narrow transmission band, the absorption spectrum of water and the reflectivity of skin, the processor storing the infiltration data in memory, and, at the time of acquiring the then-detected blood glucose signal obtaining a then-detected infiltration data, and the processor comparing the stored infiltration data to the infiltration data, and issuing an alarm via the user interface alarm if the differential therebetween stored and detected infiltration data exceeds a predetermined positional value.
